(19) United States
(12) Patent Application Publication
(10) Pub. No.: US 2019/0388406 A1
(43) Pub. Date: Dec. 26, 2019

(54) METHODS OF PREVENTION AND TREATMENT OF THROMBOSIS

(71) Applicant: Portola Pharmaceuticals, Inc., South San Francisco, CA (US)

(72) Inventor: Olga Bandman, Mountain View, CA (US)

(21) Appl. No.: 16/427,209

(22) Filed: May 30, 2019

Related U.S. Application Data
(60) Provisional application No. 62/678,951, filed on May 31, 2018.

(51) Int. Cl. A61K 31/44; A61P 7/02

(52) U.S. Cl. CPC A61K 31/44; A61P 7/02

(57) ABSTRACT

Provided herein are methods for preventing and treating thrombosis in critically ill patients or in a patient being admitted to an intensive care unit by administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban.

### CROSS-REFERENCE TO RELATED APPLICATIONS

This application claims the benefit under 35 U.S.C. § 119(e) of U.S. Provisional Application 62/678,951, filed May 31, 2018, which is hereby incorporated by reference in its entirety.

### FIELD

Provided herein are methods of preventing and treating thrombosis in critically ill patients or in patients administered to an intensive care unit.

### BACKGROUND

U.S. Pat. Nos. 6,376,515 B2, 6,835,739 B2, 8,404,724, 8,518,977, 8,524,907, 8,557,852, 8,946,269, 8,394,964, and 9,555,023, the contents of which are incorporated herein by reference, disclose a specific factor Xa inhibitor compound, betrixaban, having the chemical name of N-(5-chloropyridin-2-yl)-2-(4-(N,N-dimethylcarbamimidoyl)benzamido)-5-methoxybenzamide (betrixaban), and chemical structure of:

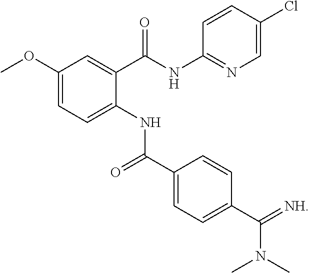

### SUMMARY

The present disclosure provides methods and compositions for the prevention and/or treatment of thrombosis in a critically ill patient or in a patient admitted to an intensive care unit. In some embodiments, the method comprises administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban. In some embodiments, the thrombosis is venous thrombosis.

In some embodiments, provided is a method for prophylaxis of venous thromboembolism (VTE) in a critically ill patient or in a patient who is admitted to an intensive care unit for an acute medical illness, the method comprises administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban. In some embodiments, the patient is at risk for thromboembolic complications. In some embodiments, the patient has moderate or severe restricted mobility and other risk factors for VTE.

In some embodiments, provided is a method for the prevention and/or treatment of thrombosis in an intensive care unit comprising administering betrixaban, or a pharmaceutically acceptable salt thereof, or a crystalline polymorph of betrixaban, or a crystalline polymorph of a pharmaceutically acceptable salt of betrixaban to a patient being cared for in the intensive care unit.

In some embodiments, provided is a method for prophylaxis of venous thromboembolism (VTE) in a patient being cared for in an intensive care unit comprising administering betrixaban, or a pharmaceutically acceptable salt thereof, or a crystalline polymorph of betrixaban, or a crystalline polymorph of a pharmaceutically acceptable salt of betrixaban to the patient.

In some embodiments, provided is use of betrixaban, or a pharmaceutically acceptable salt thereof, or a crystalline polymorph of betrixaban, or a crystalline polymorph of a pharmaceutically acceptable salt of betrixaban, for prophylaxis of venous thromboembolism (VTE) in an intensive care unit for a patient being cared for or obtaining medical treatment therein.

In some embodiments, the patient is at risk of developing a venous thromboembolic disease. In some embodiments, the patient suffers from one or more of (a) acutely decompensated heart failure, (b) acute respiratory failure, (c) acute infection without septic shock, (d) an acute rheumatic disorder or (e) cancer. In some embodiments, the patient suffers from decreased mobility.

In some embodiments, betrixaban is in the form of a pharmaceutically acceptable salt. In some embodiments, the pharmaceutically acceptable salt of betrixaban is the maleate salt. In some embodiments, the pharmaceutically acceptable salt of the crystalline polymorph of betrixaban is betrixaban maleate. In some embodiments, betrixaban is administered orally. Betrixaban can be administered in an amount of about 20 mg to about 80 mg per day, or from about 40 mg to about 80 mg per day, and in some embodiments, 80 mg per day, when administered with food. In some embodiments, a 160-mg loading dose is used prior to the daily administration. In some embodiments, the 160-mg loading dose is administered once in a single dose. In some embodiments, the initial single dose of 160 mg is followed by 80 mg once daily. In some embodiments, administration of betrixaban, or a pharmaceutically acceptable salt thereof, or a crystalline polymorph of betrixaban, or a crystalline polymorph of a pharmaceutically acceptable salt of betrixaban continues after the patient is discharged from the intensive care unit. In some embodiments, administration of betrixaban, or a pharmaceutically acceptable salt thereof, or a crystalline polymorph of betrixaban, or a crystalline polymorph of a pharmaceutically acceptable salt of betrixaban continues after the patient is discharged from the hospital. In some embodiments, the duration of treatment is 35 to 42 days. In some embodiments, betrixaban is administered in capsules comprising 80 mg or 40 mg betrixaban or pharmaceutically acceptable salt thereof, such as betrixaban maleate salt, or a crystalline polymorph of betrixaban, or a crystalline polymorph of a pharmaceutically acceptable salt of betrixaban.

### BRIEF DESCRIPTION OF THE DRAWINGS

FIG. 1 shows the risk-benefit of betrixaban versus enoxaparin in VTE and major bleeding among critically ill patients in the overall population.

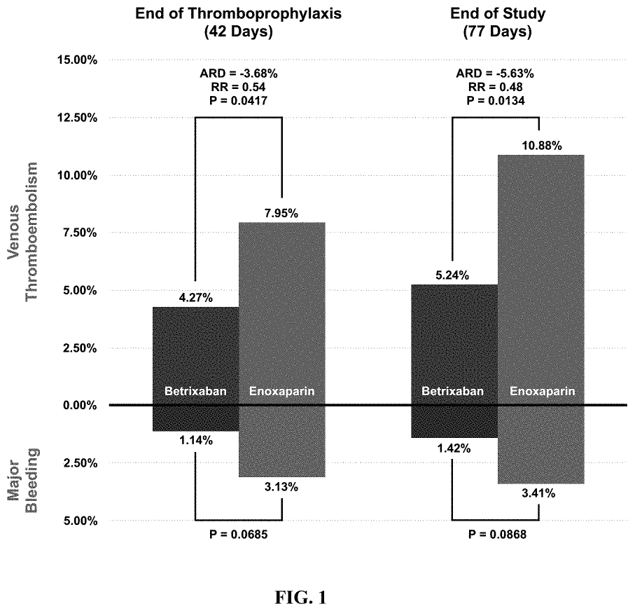

FIG. 2 shows the risk-benefit of betrixaban versus enoxaparin in VTE and major bleeding among critically ill patients in the full-dose stratum.

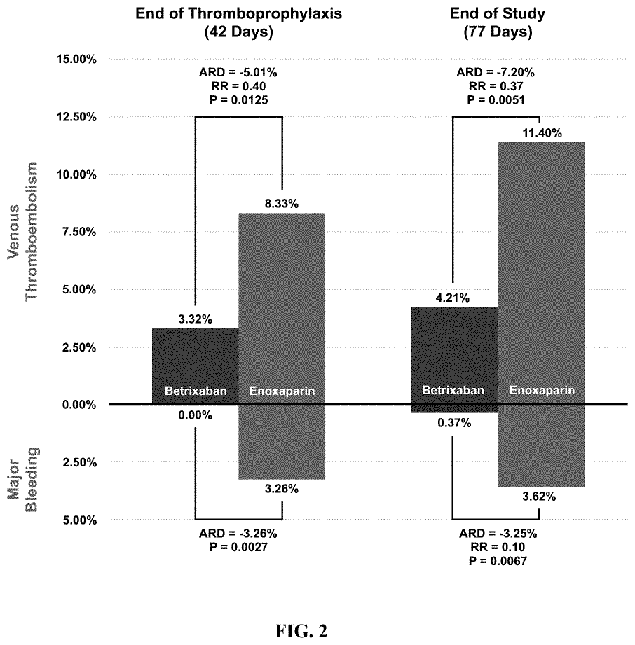

### DETAILED DESCRIPTION

### I. Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

As used in the specification and claims, the singular form “a”, “an” and “the” include plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, stereoisomers, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts of the compound (or deuterated analog, stereoisomer, or mixture of stereoisomers) with inorganic acids or organic acids. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “treatment” or “treating” means any treatment of a disease or disorder in a subject, such as a mammal, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, condition, disease, or disorder, thereby substantially averting onset of the ailment, condition, disease, or disorder.

As used herein, the term “suppressing” refers to prophylactic treatment of a patient in need thereof. The prophylactic treatment can be accomplished by providing an appropriate dose of a therapeutic agent to a subject suffering from an underlying cause of an ailment, condition, disease, or disorder, but substantially averting onset of symptoms of the ailment, condition, disease, or disorder.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein.

The term “therapeutically effective amount” refers to that amount of betrixaban, typically delivered as a pharmaceutical composition, that is sufficient to effect treatment, as defined herein, when administered to a subject in need of such treatment. In some embodiments, the effective amount of betrixaban is about 20 mg to about 80 mg per day, or from about 40 mg to about 80 mg per day, and in some embodiments, about 80 mg per day. In some embodiments, the effective amount of betrixaban comprises a 160-mg loading dose administered prior to the daily administration. In some embodiments, the initial single dose of 160 mg is followed by 80 mg once daily.

As used herein, the term “a critically ill patient” refers to a patient who is admitted to the intensive care unit (ICU) and stays in the ICU for at least 12 hours. In some embodiments, the critically ill patient stays in the ICU for at least 24 hours. In some embodiments, the critically ill patient stays in the ICU for at least two days, three days or four days. In some embodiments, the critically ill patient remains admitted to the hospital after being discharged from the ICU.

The “critically ill” or “critically ill patient” may have one or more conditions including, but are not limited to:
1. Acutely decompensated heart failure, New York Heart Association (NYHA) class III or IV;
2. Acute respiratory failure without the need for prolonged (<=2 days) respiratory support;
3. Acute infection without septic shock;
4. Acute rheumatic disorders (including acute lumbar pain, sciatica, vertebral compression, acute arthritis of the legs, or an episode of inflammatory bowel disease) or
5. Cancer.

Risk factors for venous thromboembolism (VTE) include, but are not limited to:
a) Age>75 years;
b) Previous history of VTE that required anticoagulant therapy;
c) Expected marked immobilization>=3 days (Level 1−bedrest without bathroom privileges);
d) Obesity (Body Mass Index (BMI)>30 for men or 28.6 for women);
e) Varicose veins or chronic venous insufficiency;
f) Lower extremity paresis;
g) Central venous catheterization;
h) Hormone therapy (antiandrogen, estrogen or selective estrogen receptor modulators (SERMs));
i) Chronic heart failure;
j) Chronic respiratory failure;
k) Active collagen vascular disease;
l) Acute infectious disease contributing to current hospitalization;
m) Erythropoeisis stimulating agents;
n) Inflammatory bowel disease;
o) Venous compression (tumor, hematoma or arterial anomaly);
p) Nephrotic syndrome; and
q) Inherited or acquired thrombophilia,
r) or a combination thereof.

In some embodiments, a critically ill patient has any one or more of conditions 1 to 4 above and has either at least two venous thromboembolism (VTE) risk factors from a) to q) as outlined above or a D-dimer result of more than two times the upper limit of normal.

As used herein, the term “thrombosis” refers to the formation of a blood clot inside a blood vessel, obstructing the flow of blood through the circulatory system. In some embodiments, the thrombosis is “venous thrombosis” which is a blood clot that forms within a vein.

As used herein, the term “patient” or “subject” refers to a human patient.

The term “about” when used before a numerical value indicates that the value may vary within reasonable range, such as ±5%, ±1%, and ±0.5%.

### Abbreviations

ARD, absolute risk difference

CI, confidence interval

DVT, deep vein thrombosis

ICU, intensive care unit

ISTH, International Society on Thrombosis and Haemostasis

LMWH, low-molecular-weight heparin

mITT, modified intention-to-treat

NNH, number needed to harm

NNT, number needed to treat

NOAC, non-vitamin K antagonist oral anticoagulant

PE, pulmonary embolism

RR, relative risk

SD, standard deviation

UFH, unfractionated heparin

ULN, upper limit of normal

VTE, venous thromboembolism

### II. Methods

The present disclosure provides methods and compositions for the prevention and/or treatment of thrombosis in a critically ill patient or in a patient admitted to an intensive care unit. In some embodiments, the method comprises administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban. In some embodiments, the thrombosis is venous thrombosis.

In some embodiments, provided is a method for prophylaxis of venous thromboembolism (VTE) in a patient who is admitted to an intensive care unit for an acute medical illness.

In some embodiments, provided is a method for prophylaxis of venous thromboembolism (VTE) in an intensive care unit comprising administering betrixaban, or a pharmaceutically acceptable salt thereof, or a crystalline polymorph of betrixaban, or a crystalline polymorph of a pharmaceutically acceptable salt of betrixaban to a patient being cared for in the intensive care unit.

Surprisingly, as compared with low molecular weight heparin, such as enoxaparin, betrixaban is not only significantly more effective in reducing the risk of VTE in critically ill patients or patients admitted to an intensive care unit, but also safer in that it was found to be associated with a lower risk of major bleeding among these patients. Also surprisingly, betrixaban, the first and, to date, only anticoagulant for the prophylaxis of venous thromboembolism (VTE) in adult patients hospitalized for an acute medical illness, was found to be even more effective and safer in the critically ill patients who are admitted to an intensive care unit as compared to the general hospitalized medically ill patient population.

In some embodiments, the patient is at risk for thromboembolic complications.

In some embodiments, the patient has moderate or severe restricted mobility and other risk factors for VTE.

In some embodiments, a critically ill patient or the patient being admitted to or cared for in an intensive care unit is one that is at risk of developing a venous thromboembolic disease. In some embodiments, the patient suffers from one or more of (a) acutely decompensated heart failure, (b) acute respiratory failure, (c) acute infection without septic shock, (d) an acute rheumatic disorder or (e) cancer. In some embodiments, the patient suffers from decreased mobility.

In some embodiments, the acutely decompensated heart failure is New York Heart Association (NYHA) class III or IV. In some embodiments, the acute respiratory failure is without the need for prolonged (<=2 days) respiratory support. In some embodiments, the acute infection is without septic shock. In some embodiments, the patient suffers from one or more acute rheumatic disorders (including acute lumbar pain, sciatica, vertebral compression, acute arthritis of the legs, or an episode of inflammatory bowel disease).

In some embodiments, the disclosure provides a method for the prevention or treatment of thrombosis in a critically ill patient, comprising administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban.

In some embodiments, the patient is at risk of developing a venous thromboembolic disease. In another embodiment, the patient suffers from decreased mobility. In another embodiment, the thrombosis is venous thrombosis.

In some aspects, thrombosis is a feature of an underlying disease or condition. Non-limiting examples of such disease or condition include acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, thromboembolic stroke, systemic embolism, ischemic stroke, venous thromboembolism, atrial fibrillation, non-valvular atrial fibrillation, atrial flutter, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, thrombotic complications associated with the fitting of prosthetic devices, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

In some embodiments, the disease or condition is selected from the group consisting of embolic stroke, thrombotic stroke, venous thrombosis, deep venous thrombosis, acute coronary syndrome, and myocardial infarction.

In some embodiments, the therapeutically effective amount is an aggregate daily dose of about 20 mg to about 80 mg, or about 40 mg to about 80 mg of betrixaban, or a pharmaceutically acceptable salt thereof, or a crystalline polymorph of betrixaban, or a crystalline polymorph of a pharmaceutically acceptable salt of betrixaban, such as a maleate salt. In some embodiments, betrixaban is administered when the patient is in a fasted state. In some embodiments, the therapeutically effective amount of betrixaban is about 80 mg per day, with food. In some embodiments, the therapeutically effective amount of betrixaban comprises a 160-mg loading dose administered prior to the daily administration. In some embodiments, the initial single dose of 160 mg is followed by 80 mg once daily. In some embodiments, the duration of treatment is 35 to 42 days. In some embodiments, betrixaban is administered in capsules comprising 80 mg or 40 mg betrixaban or pharmaceutically acceptable salt thereof, such as betrixaban maleate salt. In some embodiments, the daily dose may be administered once, twice or three times daily. In some embodiments, the dosage is an aggregate daily dose of about 20, 30, 40, 50, 60, 70, or 80 mg and may be administered once, twice or three times daily, and in some embodiments, once or twice daily. In some embodiments, the dosage is an aggregate daily dose of about 20, 40 or 80 mg and may be administered once or twice daily, and in some embodiments, once daily. In some embodiments, the amount refers to the amount of betrixaban free base. In some embodiments, when a salt of betrixaban is administered, the amount of the salt provides betrixaban free base in an amount as described herein.

In some embodiments, a P-glycoprotein (Pgp) inhibitor is concomitantly administered to the patient. It is discovered that Pgp inhibitor increases the exposure of betrixaban, and therefore co-administration with a Pgp inhibitor enables betrixaban to be administered at a subtherapeutic dose, or alternatively a synergistically effective dose. In one aspect, when used together with a Pgp inhibitor, betrixaban is administered at about 50 mg, at about 40 mg, or alternatively about 35, or 30, or 25, or 20, or 15, or 10 mg daily, or any ranges between any two of the values, end points inclusive. The same amount is applicable to patients who experience kidney insufficiency.

Examples of Pgp inhibitors include but are not limited to amiodarone, ketoconazole, clarithromycin, verapamil, diltiazem, cyclosporine, quinidine, erythromycin, itraconazole, ivermectin, mefloquine, nifedipine, ofloxacin, propafenone, ritonavir, tacrolimusvalspodar (PSC-833), zosuquidar (LY-335979), elacridar (GF120918), HM30181AK, R101933, and R102207, or a pharmaceutically acceptable salt thereof.

In another embodiment, betrixaban is administered with food; that is, betrixaban is administered together with food, or within a short period before or after food consumption. A “short period” as used here, refers to an amount of time that is about 30 minutes or shorter, or alternatively about 25, 20, 15, 20, or 5 minutes or shorter.

### III. Betrixaban, its Salts and Crystalline Polymorph Form

Betrixaban has the chemical name of N-(5-chloropyridin-2-yl)-2-(4-(N,N-dimethylcarbamimidoyl)benzamido)-5-methoxybenzamide and has been disclosed as Example 206 in U.S. Pat. Nos. 6,376,515 and 6,835,739, both of which are incorporated by reference in their entirety herein. Further descriptions of salts and polymorphs of salts of betrixaban can be found in U.S. Pat. Nos. 7,598,276 and 8,946,269, both of which are incorporated by reference in their entirety herein.

In some embodiments, the salt of betrixaban is a maleate salt. The maleate salt be formed by protonating one or more nitrogen atoms of betrixaban. In some embodiments, the amidino nitrogen (═NH) of betrixaban is protonated (═NH₂⁺) to form the salt. In some embodiments, the therapeutically effective amount is an aggregate daily dose of 80 mg and in some embodiments, betrixaban is administered in the form of a salt, for example the maleate salt that provides about 80 mg of betrixaban free base.

In some embodiments, the maleate salt of betrixaban is represented by Formula I:

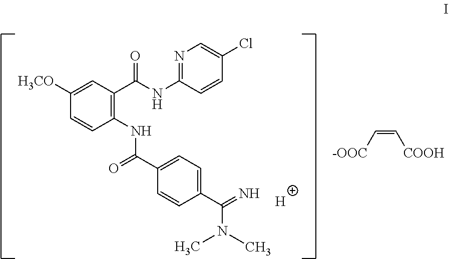

This is also referred to herein as betrixaban maleate. In some embodiments, the therapeutically effective amount is an aggregate daily dose of about 20 to about 80 mg of betrixaban maleate. In another embodiment, the aggregate daily dose is about 40 mg, 60 mg, or 80 mg aggregate of betrixaban maleate. In some embodiments, the therapeutically effective amount of betrixaban maleate provides an aggregate daily dose of about 20 to about 80 mg of betrixaban free base. In another embodiment, the aggregate daily dose of betrixaban maleate provides about 40 mg, 60 mg, or 80 mg aggregate of betrixaban free base.

In another embodiment, the salt of betrixaban has a crystalline polymorph form (Form I). Form I exhibits a powder X-ray diffraction pattern having at least four, at least six or at least eight of the following approximate characteristic peak locations: 4.9, 9.7, 13.8, 14.1, 15.2, 17.6, 18.5, 20.8, 21.6, 22.7, 24.1, 26.3, 26.8 degrees 2θ. In still another embodiment, the powder X-ray diffraction pattern has approximate characteristic peak locations of 4.9, 9.7, 11.8, 13.8, 14.1, 15.2, 17.6, 18.5, 19.9, 20.8, 21.6, 22.7, 24.1, 25.0, 26.3, 26.8 degrees 2θ. It is contemplated that the approximate characteristic XRPD peaks will have a deviation of about 0.2, 0.1, or 0.05 degrees 2θ. In some embodiments, the characteristic XRPD peaks will have an uncertainty of ±0.2, ±0.1, or ±0.05 degrees 2θ. Form I is further described in U.S. Pat. No. 7,598,276, which is incorporated by reference in its entirety herein. In some embodiments, Form I has a melting point of 201° C.

In another embodiment, the salt of betrixaban has a crystalline polymorph form, Form II. In some embodiments, Form II is an anhydrate. In some embodiments, Form II is characterized by properties including one or more of the following as described in details herein:
its X-ray powder diffraction pattern (XRPD);
its infrared spectrum (IR);
its differential scanning calorimetry (DSC);
its thermogravimetric analysis (TGA);
its vapor sorption curve,
solid state NMR, and
crystal structure, such as unit cell structure.

In some embodiments, Form II exhibits an X-ray powder diffraction pattern having the following approximate characteristic peak locations: 5.0, 9.7, 10.1, 15.3, 17.5, and 19.6 degrees 2θ. In another embodiment, the X-ray powder diffraction pattern has at least four, six, eight or ten of the approximate characteristic peak locations of 5.0, 9.7, 10.1, 14.6, 15.3, 17.5, 18.0, 18.7, 19.2, 19.6, 22.0, 22.6, 23.0, 23.7, 24.5, 26.5, 26.9, 29.2, 29.5, 30.4 and 35.0 degrees 2θ. In another embodiment, the X-ray powder diffraction pattern has at least four, six, eight or ten of the approximate characteristic peak locations of 5.0, 9.5, 9.7, 10.1, 14.6, 15.3, 17.5, 18.0, 18.7, 19.2, 19.6, 22.0, 22.6, 23.0, 23.7, 24.5, 26.5, 26.9, 29.2, 29.5, 30.4 and 35.0 degrees 2θ. In another embodiment, the X-ray powder diffraction pattern has at least four, six, eight or ten of the approximate characteristic peak locations of 15.3, 5.0, 10.1, 17.5, 9.7, 19.6, 24.5, 18.6, 18.0, 14.5, 22.6, 22.9, 23.0, 22.1, 29.2, 26.5, 24.8, 18.3, and 21.6 degrees 2θ. It is contemplated that the approximate characteristic XRPD peaks will have a deviation of up to about 0.2, 0.1, or 0.05 degrees 2θ. In some embodiments, the characteristic XRPD peaks will have an uncertainty of ±0.2, ±0.1, or ±0.05 degrees 2θ. Form II is further described in U.S. Pat. No. 8,946,269, which is incorporated by reference in its entirety herein.

In some embodiments, Form II is an anhydrous crystalline form. In some embodiments, it is a white solid with high melting point (213° C.). It absorbs up to 1% water at 95% RH.

In another aspect, there is provided a crystalline polymorph Form III of the maleate salt of betrixaban. In some embodiments, Form III is characterized by properties including one or more of the following as described in details herein:
its X-ray powder diffraction pattern (XRPD);
its infrared spectrum (IR);
its differential scanning calorimetry (DSC);
its thermogravimetric analysis (TGA);
its vapor sorption curve;
solid state NMR, and
crystal structure, such as unit cell structure.

In some embodiments, Form III exhibits an X-ray powder diffraction pattern having at least the following approximate characteristic peak locations 15.1, 2.2, 4.9, 17.4, 10.0, and 22.4 degrees 2θ. In some embodiments, the X-ray powder diffraction pattern is characterized with peaks having a relative intensity of 10% or more: 15.1, 2.2, 4.9, 17.4, 10.0, 22.4, 26.5, and 2.9 degrees 2θ. In another embodiment, the X-ray powder diffraction pattern has at least three, six, or eight, or ten, or all of the approximate characteristic peak locations selected from 15.1, 2.2, 4.9, 17.4, 10.0, 22.4, 26.5, 2.9, 24.6, 19.4, 24.2, 16.3, 20.7, 22.9, 29.0, 9.6, 18.0, 18.5, 29.3, 22.0, and 30.3 degrees 2θ. In another embodiment, the X-ray powder diffraction pattern has at least four, six, eight, ten or all of the approximate characteristic peak locations of 15.1, 2.2, 4.9, 17.4, 10.0, 22.4, 26.5, 2.9, 24.6, 19.4, 24.2, 16.3, 20.7, 22.9, 29.0, 9.6, 18.0, 18.5, and 29.3 degrees 2θ. It is contemplated that the approximate characteristic XRPD peaks will have a deviation of up to about 0.2, 0.1, or 0.05 degrees 2θ. In some embodiments, the characteristic XRPD peaks will have an uncertainty of ±0.2, ±0.1, or ±0.05 degrees 2θ. Form III is further described in U.S. Pat. No. 8,946,269, which is incorporated by reference in its entirety herein.

In some embodiments, Form III is a hydrate. In some embodiments, Form III is a hemihydrate. In some embodiments, the Form III is a channel hydrate.

In some embodiments, Form III is a hemihydrate with two independent salt pairs of betrixaban and maleic acid in a crystallographic asymmetric unit. The two cations have dissimilar overall conformations arising from a substantial rotation about the N1-C11 bond of approximately 100 degrees. In some embodiments, the crystal structure is characterized by a unit cell structure with the following cell parameters at 100 K:

| | | |
|---|---|---|
| a = 8.2369(4) Å | α = 107.045(4)° | V = 2675.7(2) Å³ |
| b = 18.3639(9) | β = 93.758(4) | Space group = P1, #2 |
| c = 18.5623(9) | γ = 91.459(4) | Z = 2 |

Betrixaban can be prepared according to methods described in U.S. Pat. Nos. 6,376,515 and 7,598,276, and U.S. patent application Ser. No. 12/969,371, filed Dec. 15, 2010, now U.S. Pat. No. 8,394,964, all of which are hereby incorporated by reference in their entirety. Preparation of the maleate salt of betrixaban and Form I is described in U.S. Pat. No. 7,598,276. Preparation of Forms II and III is described in U.S. Patent Application Publication No. 2012/0071519, and U.S. Pat. No. 8,946,269.

### IV. Formulations

In another aspect, provided a unit dose formulation comprising betrixaban in an amount of about 20 mg to about 80 mg, or about 80 mg of betrixaban, or a pharmaceutically acceptable salt thereof, or a crystalline polymorph of betrixaban, or a crystalline polymorph of a pharmaceutically acceptable salt of betrixaban, such as betrixaban maleate, or alternatively about 40 mg, or 20 mg of betrixaban, or a pharmaceutically acceptable salt thereof, or a crystalline polymorph of betrixaban, or a crystalline polymorph of a pharmaceutically acceptable salt of betrixaban, such as betrixaban maleate. In some embodiments, the aggregate daily dose is formulated for administration to the patient once or twice daily. In some embodiments, the unit dose formulation further comprises a pharmaceutically acceptable carrier. In some embodiments, the formulation further includes a P-glycoprotein inhibitor. In one such aspect, the amount of betrixaban in the formulation is a synergistically effective amount. In some embodiments, the formulation is a capsule for oral administration comprising about 40 mg or about 80 mg of betrixaban, or a pharmaceutically acceptable salt thereof, or a crystalline polymorph of betrixaban, or a crystalline polymorph of a pharmaceutically acceptable salt of betrixaban, such as betrixaban maleate. In some embodiments, the capsule further comprises glucose monohydrate, croscarmellose sodium and magnesium stearate. In some embodiments, the capsule is hard gelatin capsule.

The compositions of this invention may be in the form of tablets, capsules, lozenges, or elixirs for oral administration, suppositories, sterile solutions or suspensions or injectable administration, and the like, or incorporated into shaped articles. The method of administration will vary from subject to subject and be dependent upon such factors as the type of mammal being treated, its sex, weight, diet, concurrent medication, overall clinical condition, the particular compounds and/or salts employed, the specific use for which these compounds and/or salts are employed, and other factors which those skilled in the medical arts will recognize.

Capsules useful in the present invention can be prepared using conventional and known encapsulation techniques, such as that described in Stroud et al., U.S. Pat. No. 5,735,105. The capsule is typically a hollow shell of generally cylindrical shape having a diameter and length sufficient so that the pharmaceutical solution compositions containing the appropriate dose of the active agents fit inside the capsule. The exterior of the capsules can include plasticizer, water, gelatin, modified starches, gums, carrageenans, and mixtures thereof. Those skilled in the art will appreciate what compositions are suitable.

In addition to the active agents, tablets useful in the present invention can comprise fillers, binders, compression agents, lubricants, disintegrants, colorants, water, talc and other elements recognized by one of skill in the art. The tablets can be homogeneous with a single layer at the core, or have multiple layers in order to realize preferred release profiles. In some instances, the tablets of the instant invention may be coated, such as with an enteric coating. One of skill in the art will appreciate that other excipients are useful in the tablets of the present invention.

Formulations suitable for delivery through a nasogastric tube are also contemplated. Administration using a nasogatric tube is useful considering that the medically ill patient may not be able to receive betrixaban orally. In some aspects, betrixaban is mixed with nutritionally ingredients that the medically ill patient takes as food supplement.

Lozenges useful in the present invention include an appropriate amount of the active agents as well as any fillers, binders, disintegrants, solvents, solubilizing agents, sweeteners, coloring agents and any other ingredients that one of skill in the art would appreciate is necessary. Lozenges of the present invention are designed to dissolve and release the active agents on contact with the mouth of the patient. One of skill in the art will appreciate that other delivery methods are useful in the present invention.

Formulations of this invention are prepared for storage or administration by mixing active agents having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro Ed. 1985). Such materials are nontoxic to the recipients at the dosages and concentrations employed, and include buffers such as phosphate, citrate, acetate and other organic acid compounds and/or salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium, and/or nonionic surfactants such as Tween, Pluronics or polyethyleneglycol.

In some embodiments, dosage formulations to be used for therapeutic administration are sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be between 3 and 11, or from 5 to 9, or from 7 to 8. It will be understood that use of certain of the foregoing excipients, carriers, or stabilizers may result in the formation of cyclic polypeptide compounds and/or salts. Route of administration may be by injection, such as intravenously (bolus and/or infusion), subcutaneously, intramuscularly, or colonically, rectally, nasally or intraperitoneally. Other dosage forms such as suppositories, implanted pellets or small cylinders, aerosols, oral dosage formulations (such as tablets, capsules and lozenges) and topical formulations such as ointments, drops and dermal patches may be used. The sterile membranes may be desirably incorporated into shaped articles such as implants which may employ inert materials such as biodegradable polymers or synthetic silicones, for example, Silastic, silicone rubber or other polymers commercially available.

The compositions of this invention may be in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compositions of this invention may also be delivered by the use of antibodies, antibody fragments, growth factors, hormones, or other targeting moieties, to which the salt molecules are coupled. The compositions of this invention may also be coupled with suitable polymers as targetable drug carriers. Such polymers can include polyvinylpyrrolidinone, pyran copolymer, polyhydroxy-propyl-methacrylamide-phenol, polyhydroxyethyl-aspartamide-phenol, or polyethyleneoxide-polylysine substituted with palmitoyl residues. Furthermore, compositions of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

In some embodiments, an amiodarone tablet comprises amiodarone hydrochloride, lactose monohydrate, magnesium stearate, povidone, pregelatinized corn starch, sodium starch glycolate, steric acid, and optionally one or more coloring agents.

### EXAMPLES

The present technology is further defined by reference to the following examples. It will be apparent to those skilled in the art that many modifications, both to compositions and methods, may be practiced without departing from the scope of the current invention.

A multi-center, double-blind, randomized controlled trial enrolled 7,513 patients hospitalized for an acute medical illness with the following eligibility criteria: (1) hospitalization for acute medical illness, including heart failure, respiratory failure, infection, ischemic stroke, or rheumatic disorder; (2) age≥75 years, age 60 to 74 years with D-dimer≥2×the upper limit of normal (ULN), or age 40 to 59 years with D-dimer≥2×ULN and history of VTE or cancer; (3) anticipated severe immobilization for ≥24 hours followed by moderate or severe immobilization for 3 or more days; and (4) anticipated hospitalization for 3 or more days. Patients were randomly allocated in a 1:1 ratio to extended-duration betrixaban (35 to 42 days) or standard-duration enoxaparin (10±4 days).

The present study is a post hoc analysis of the effect of betrixaban in critically ill patients admitted to the intensive care unit (ICU) during the index hospitalization and received extended-duration betrixaban. The study population included in the present analysis was subjects admitted to the ICU during the index hospitalization event.

The primary efficacy endpoint was the occurrence of VTE, defined as a composite of asymptomatic proximal DVT, symptomatic DVT, non-fatal pulmonary embolism (PE), or VTE-related mortality. Safety endpoints were the occurrence of major bleeding and clinically relevant non-major bleeding, defined according to the International Society on Thrombosis and Haemostasis (ISTH) criteria. (Schulman S, Kearon C, Subcommittee on Control of Anticoagulation of the S, Standardization Committee of the International Society on T, Haemostasis. Definition of major bleeding in clinical investigations of antihemostatic medicinal products in non-surgical patients. J Thromb Haemost. 2005; 3:692-694; and Kaatz S, Ahmad D, Spyropoulos AC, Schulman S, Subcommittee on Control of A. Definition of clinically relevant non-major bleeding in studies of anticoagulants in atrial fibrillation and venous thromboembolic disease in non-surgical patients: communication from the SSC of the ISTH. J Thromb Haemost. 2015; 13:2119-2126.) All events were adjudicated by an independent clinical events committee blinded to thromboprophylaxis allocation.

Baseline characteristics of study participants were compared between treatment arms using the chi-squared test for categorical variables and one-way analysis of variance (ANOVA) for continuous variables, respectively. We analyzed efficacy using a modified intention-to-treat (mITT) population, which included subjects who received at least one dose of study medication and had data on one or more of the efficacy components. The bleeding risk of thromboprophylaxis was analyzed with the actual regimen received in the safety population, which included all randomized patients who received at least one dose of study medication. In addition, treatment effect was also analyzed in the full-dose stratum (betrixaban 80 mg daily vs. enoxaparin 40 mg daily), which comprised subjects who had a creatinine clearance of ≥30 mL/min and without concomitant use of strong P-glycoprotein inhibitors. Absolute risk difference and relative risk of betrixaban vs. enoxaparin group were calculated and compared using the chi-squared test. The Breslow-Day test of homogeneity was performed to assess the effect modification by ICU admission on the thromboprophylaxis. All analyses were performed using two-sided tests at a significance level of 0.05 with SAS software version 9.4 (SAS Institute, Inc., North Carolina, United States).

A total of 703 critically ill medical patients were admitted to the intensive care unit (ICU) and included in the analysis. The full-dose stratum included 547 (77.8%) patients. Baseline characteristics were generally balanced (Table 1), except that patients in the betrixaban group were slightly older than those in the enoxaparin group (p=0.041). Between treatment arms, there was no difference with respect to sex, race, body mass index, creatinine clearance, duration of ICU stay or hospital stay, thromboprophylaxis use within 96 hours, acute medical illness, VTE risk factor, IMPROVE VTE risk score, or baseline D-dimer level.

TABLE 1
Baseline characteristics stratified by thromboprophylaxis
among critically ill patients

| | Betrixaban | | Enoxaparin | | |
|---|---|---|---|---|---|
| Characteristic | (N = 351) | | (N = 352) | | p-value |
| Age, mean (SD) - years | 75.9 | (8.4) | 74.6 | (8.7) | 0.041 |
| Male sex, n (%) | 153 | (43.6) | 159 | (45.2) | 0.67 |
| Race, n (%) | | | | | 0.72 |
| White | 344 | (98.0) | 341 | (96.9) | |
| Black/African American | 3 | (0.9) | 3 | (0.9) | |
| Asian | 1 | (0.3) | 2 | (0.6) | |
| Others | 3 | (0.9) | 6 | (1.7) | |
| Body mass index, mean | 29.5 | (6.5) | 29.4 | (6.2) | 0.88 |
| (SD) - kg/m² | | | | | |
| Creatinine clearance, n (%) | | | | | 0.85 |
| <30 mL/min | 15 | (4.3) | 13 | (3.7) | |
| ≥30 to <60 mL/min | 153 | (43.6) | 147 | (41.8) | |
| ≥60 to <90 mL/min | 112 | (31.9) | 112 | (31.8) | |
| ≥90 mL/min | 71 | (20.2) | 80 | (22.7) | |
| Duration of ICU stay, | 6.2 | (4.7) | 6.6 | (5.7) | 0.31 |
| mean (SD) - days | | | | | |
| Duration of hospital | 13.3 | (8.5) | 14.4 | (10.2) | 0.13 |
| stay, mean (SD) - days | | | | | |
| Previous | 202 | (57.5) | 214 | (60.8) | 0.38 |
| thromboprophylaxis ≤96 | | | | | |
| hours, n (%) | | | | | |
| Acute medical | | | | | 0.52 |
| condition, n (%) | | | | | |
| Acute heart failure | 173 | (49.3) | 151 | (42.9) | |
| Respiratory failure | 30 | (8.5) | 37 | (10.5) | |
| Infection | 61 | (17.4) | 64 | (18.2) | |
| Rheumatic disorder | 13 | (3.7) | 14 | (4.0) | |
| Ischemic stroke | 74 | (21.1) | 86 | (24.4) | |
| VTE risk factor, n (%) | | | | | |
| History of VTE | 18 | (5.1) | 26 | (7.4) | 0.22 |
| Known thrombophilia * | 1 | (0.3) | 1 | (0.3) | 1.00 |
| Current lower-limb | 34 | (9.7) | 29 | (8.2) | 0.50 |
| paralysis | | | | | |
| Active cancer | 15 | (4.3) | 9 | (2.6) | 0.21 |
| Age > 60 years | 331 | (94.3) | 330 | (93.8) | 0.76 |
| IMPROVE VTE risk | 2.4 | (0.9) | 2.4 | (0.9) | 0.99 |
| score, mean (SD) | | | | | |
| D-dimer, mean | 2.26 | (3.10) | 2.27 | (3.06) | 0.98 |
| (SD) - μg/mL | | | | | |

Among critically ill patients, at the end of extended thromboprophylaxis through 42 days, betrixaban reduced the risk of VTE by 46% compared with enoxaparin (mITT population: 4.27% vs. 7.95%, p=0.0417, NNT=28; full-dose stratum: 3.32% vs. 8.33%, p=0.0125, NNT=20) (Table 2). The treatment effect of betrixaban persisted until the end of study through 77 days, with a relative risk reduction of 52% (mITT population: 5.24% vs. 10.88%, p=0.0134, NNT=18; full-dose stratum: 4.21% vs. 11.40%, p=0.0051, NNT=14). Risk reduction by betrixaban in the VTE components (i.e., asymptomatic proximal DVT, symptomatic DVT, non-fatal PE, and VTE-related death) did not reach statistical difference.

TABLE 2
The risk of VTE among critically ill patients

| | | | | | | |
|---|---|---|---|---|---|---|
| | | | ARD | RR | | NNT or |
| Population | Betrixaban | Enoxaparin | (95% CI) | (95% CI) | p-value | NNH |

End of extended thromboprophylaxis through 42 days

| | | | | | | |
|---|---|---|---|---|---|---|
| mITT population | 15/351 | 28/352 | −3.68% | 0.54 | 0.0417 | 28 |
| | (4.27%) | (7.95%) | (−0.15% to −7.21%) | (0.29 to 0.99) | | |
| Full-dose stratum | 9/271 | 23/276 | −5.01% | 0.40 | 0.0125 | 20 |
| | (3.32%) | (8.33%) | (−1.12% to −8.91%) | (0.19 to 0.85) | | |

End of study through 77 days

| | | | | | | |
|---|---|---|---|---|---|---|
| mITT population | 15/286 | 31/285 | −5.63% | 0.48 | 0.0134 | 18 |
| | (5.24%) | (10.88%) | (−1.19% to −10.08%) | (0.26 to 0.87) | | |
| Full-dose stratum | 9/214 | 26/228 | −7.20% | 0.37 | 0.0051 | 14 |
| | (4.21%) | (11.40%) | (−2.27% to −12.12%) | (0.18 to 0.77) | | |

Among non-critically ill patients, betrixaban significantly reduced VTE risk by 23% at the end of extended thromboprophylaxis and by 24% at the end of study. ICU admission did not significantly modify the effect of betrixaban on VTE risk at the end of thromboprophylaxis (p=0.27 for mITT population and p=0.15 for full-dose stratum) as well as at the end of study (p=0.14 for mITT population and p=0.09 for full-dose stratum).

In the overall safety population, the risk of major bleeding was numerically lower but not statistically different in the betrixaban group compared with enoxaparin group (after discontinuation: 1.14% vs. 3.13%, p=0.0685; end of study: 1.42% vs. 3.41%, p=0.0868) (Table 3).

TABLE 3
The risk of major bleeding among critically ill patients

| | | | | | | |
|---|---|---|---|---|---|---|
| | | | ARD | RR | | NNT or |
| Population | Betrixaban | Enoxaparin | (95% CI) | (95% CI) | p-value | NNH |

Through 7 days after discontinuation of study medication

| | | | | | | |
|---|---|---|---|---|---|---|
| Safety population | 4/351 | 11/352 | −1.99% | 0.36 | 0.0685 | — |
| | (1.14%) | (3.13%) | (−4.12% to 0.14%) | (0.12 to 1.13) | | |
| Full-dose stratum | 0/271 | 9/276 | −3.26% | — | 0.0027 | 31 |
| | (0.00%) | (3.26%) | (−1.17% to −5.36%) | | | |

End of study through 77 days

| | | | | | | |
|---|---|---|---|---|---|---|
| Safety population | 5/351 | 12/352 | −1.98% | 0.42 | 0.0868 | — |
| | (1.42%) | (3.41%) | (−4.25% to 0.28%) | (0.15 to 1.17) | | |
| Full-dose stratum | 1/271 | 10/276 | −3.25% | 0.10 | 0.0067 | 31 |
| | (0.37%) | (3.62%) | (−0.93% to −5.57%) | (0.01 to 0.79) | | |

In the full-dose stratum, betrixaban was associated with a lower risk of major bleeding among critically ill patients, with a number-needed-to-treat of 31 (after discontinuation: 0.00% vs. 3.26%, p=0.0027; end of study: 0.37% vs. 3.62%, p=0.0067). In contrast, among non-critically ill patients, there was no difference in the rates of major bleeding between betrixaban and enoxaparin. There was a significant effect modification by ICU admission on major bleeding rates (after discontinuation: p=0.001; end of study: p=0.014).

The rates of major or clinically relevant non-major bleeding were similar between betrixaban and enoxaparin in the overall safety population (after discontinuation: 3.70% vs. 3.41%, p=0.8330; end of study: 4.27% vs. 3.69%, p=0.6940) and the full-dose stratum (after discontinuation: 3.32% vs. 3.62%, p=0.8470; end of study: 4.06% vs. 3.99%, p=0.9651) (Table 4). In contrast, among non-critically ill patients, betrixaban was associated with a greater risk of major or clinically relevant non-major bleeding, with an absolute difference of 1.00 to 1.59%. The interaction term of ICU admission was not significant in the overall safety population (after discontinuation: p=0.11; end of study: p=0.17) and the full-dose stratum (after discontinuation: p=0.28; end of study: p=0.38).

TABLE 4
The risk of major or clinically relevant non-major bleeding among critically ill patients

| | | | | | | |
|---|---|---|---|---|---|---|
| | | | ARD | RR | | NNT or |
| Population | Betrixaban | Enoxaparin | (95% CI) | (95% CI) | p-value | NNH |

Through 7 days after discontinuation of study medication

| | | | | | | |
|---|---|---|---|---|---|---|
| Safety population | 13/351 | 12/352 | 0.29% | 1.09 | 0.8330 | — |
| | (3.70%) | (3.41%) | (−2.44% to 3.03%) | (0.50 to 2.35) | | |
| Full-dose stratum | 9/271 | 10/276 | −0.30% | 0.92 | 0.8470 | — |
| | (3.32%) | (3.62%) | (−3.37% to 2.77) | (0.38 to 2.22) | | |

End of study through 77 days

| | | | | | | |
|---|---|---|---|---|---|---|
| Safety population | 15/351 | 13/352 | 0.58% | 1.16 | 0.6940 | — |
| | (4.27%) | (3.69%) | (−2.31% to 3.47%) | (0.56 to 2.40) | | |
| Full-dose stratum | 11/271 | 11/276 | 0.07% | 1.02 | 0.9651 | — |
| | (4.06%) | (3.99%) | (−3.22% to 3.37%) | (0.45 to 2.31) | | |

Although the foregoing has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for the prevention or treatment of thrombosis in a critically ill patient, comprising administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban.

2. A method for prophylaxis of venous thromboembolism (VTE) in a patient who is admitted to an intensive care unit for an acute medical illness, wherein the method comprises administering to the patient a therapeutically effective amount of betrixaban, or a pharmaceutically acceptable salt thereof, or a crystalline polymorph of betrixaban, or a crystalline polymorph of a pharmaceutically acceptable salt of betrixaban.

3. A method for prophylaxis of venous thromboembolism (VTE) in a patient being cared for in an intensive care unit comprising administering betrixaban, or a pharmaceutically acceptable salt thereof, or a crystalline polymorph of betrixaban, or a crystalline polymorph of a pharmaceutically acceptable salt of betrixaban to the patient.

4. The method of claim 1, wherein the patient is at risk of developing a venous thromboembolic disease.

5. The method of claim 1, wherein the patient suffers from one or more of (a) acutely decompensated heart failure, (b) acute respiratory failure, (c) acute infection without septic shock, (d) an acute rheumatic disorder or (e) cancer.

6. The method of claim 1, wherein the patient suffers from decreased mobility.

7. The method of claim 1, wherein betrixaban is administered to the patient once daily or twice daily.

8. The method of claim 1, wherein a pharmaceutically acceptable salt of betrixaban is administered.

9. The method of claim 8, wherein the pharmaceutically acceptable salt of betrixaban is a maleate salt.

10. The method of claim 1, wherein betrixaban, or a pharmaceutically acceptable salt thereof, or a crystalline polymorph of betrixaban, or a crystalline polymorph of a pharmaceutically acceptable salt of betrixaban, is administered in an amount of, or equivalent to, about 20 mg to about 80 mg of betrixaban free base per day.

11. The method of claim 1, wherein betrixaban, or a pharmaceutically acceptable salt thereof, or a crystalline polymorph of betrixaban, or a crystalline polymorph of a pharmaceutically acceptable salt of betrixaban, is administered in an amount of, or equivalent to, about 40 mg to about 80 mg of betrixaban free base per day.

12. The method of claim 1, wherein betrixaban, or a pharmaceutically acceptable salt thereof, or a crystalline polymorph of betrixaban, or a crystalline polymorph of a pharmaceutically acceptable salt of betrixaban, is administered with food.

13. The method of claim 7, wherein the patient receives a dose of betrixaban, or a pharmaceutically acceptable salt thereof, or a crystalline polymorph of betrixaban, or a crystalline polymorph of a pharmaceutically acceptable salt of betrixaban in an amount of, or equivalent to, about 160 mg of betrixaban free base, prior to the daily administration.

14. The method of claim 1, wherein the thrombosis is venous thrombosis.
